(19) United States
(12) Patent Application Publication
(10) Pub. No.: US 2006/0229910 A1
(43) Pub. Date: Oct. 12, 2006

(54) Truly automated online prescription process

(76) Inventors: Robert Longman, Chicago, IL (US); Sophia C. Li, Chicago, IL (US)

Correspondence Address: USA Shout Media, Inc.;Attn: Robert Longman & Sophia C. Li, Unit #145, 1749 Golf Rd., Mount Prospect, IL, 60056, US

(21) Appl. No.: 11/102,373

(22) Filed: Apr. 8, 2005

(51) Int. Cl. G06Q 10/00; A61B 5/00

(52) U.S. Cl. 705/2; 600/300

(57) ABSTRACT

The present invention provides a fresh method and system for truly automated online prescription purchases while providing improved security for issuing prescription simultaneously. Within the said online pharmacy network, both patients and doctors (M.D.s) are registered users. Doctors (M.D.s) will be verified by one of the authorities, such as the American Board of Medical Specialties (ABMS), an organization that maintains a database of physician board certification information on more than 700,000 specialty physicians representing 36 medical specialties and 88 subspecialties. ABMS contains information primary sourced from 24 recognized medical specialty boards, not self reported by physicians, and is fully recognized by key healthcare accreditation organizations for credentialing purposes. Once a doctor's status is verified, he/she has the authority to issue online prescriptions or approve patients' online prescription requests within the said online pharmacy network. Patients who wish to purchase prescription online will register with the said online pharmacy network and provide personal information such as credit card numbers, email addresses, and etc. Once a patient is registered with the said online pharmacy network, he/she has the ability to request an online prescription that will be used towards prescription purchases within the said online pharmacy network. A patient can choose from diverse payment methods and delivery preferences without obtaining or dropping off a conventional paper prescription at a pharmacy that has a physical location for pick-up. The present invention will reduce the likelihood of prescription abuse by avoiding counterfeit paper prescriptions, improve the efficiency of the prescription purchase by automating the processing of prescription writing and dropping off, permitting patients who wish to order discounted prescription drug online without adopting the existing prescription fax-in or mail-in methods.

### FIELD OF INVENTION

The present invention relates to a method and system for online electronic prescription issuing, redemption, purchasing, and delivery of pharmaceuticals.

### BACKGROUND

The ability to purchase discounted prescriptions online has always been a dream of patients and endeavors of e-commerce entrepreneurs. However, while e-businesses such as online auctions and online marketing have been flourishing over the past decade, online prescription sales are facing both regulatory and operational challenges. One of the most conspicuous perplexities facing the online pharmacy industry is the inefficiency involved with written prescriptions. Online pharmacy such as drugstore.com requires patients to either mail or fax written prescriptions, which takes up about 12 days with mailing charges covered by the patients, or direct communications between the online pharmacy and doctors, which requires additional human resource and is inconvenient for the doctors. Since a prescription is designed for the patient's safety and a method to reduce prescription medicine abuse, it is indisputable such prerequisite should be sustained. Nevertheless, the operational improvements for accelerating the prescription ordering are realistic, noteworthy, and crucial angles pertaining to future growth in the online pharmacy industry. Besides the capability to purchase discounted prescriptions online, retail pharmacy and pharmacy chains such as Osco-drugs, Walgreen's, and Costco Wholesalers have all being seeking operational improvements to ease the prescription filling process for their patients, such as easy drop-off and pick-up, auto refill, auto transfer. Though these methods have reduced the waiting period for patients to obtain prescription drug, they failed to provide truly automated prescription service for patients; most notably, patients are still required to show some degree of presence at the pharmacy to obtain their prescription drugs. Furthermore, most of the written prescriptions are vulnerable to prescription abuses since they are not verified by pharmacies at the time of the prescription filling and the circumstance for anyone to obtain prescription pads over the web is relaxed; hence, the challenge to eliminate prescription abuse becomes a convoluted and mounting task. Furthermore, as the news of Medicare is expected to face insolvency by the year 2020, it becomes increasingly urgent for patients and lawmakers alike to find ways to obtain cheap prescription drugs, while simultaneously protect patients from defective and harmful pharmaceuticals. The current invention provides a fresh method for a truly automated prescription writing and filling process. It not only drastically reduces the possibility for prescription abuse, it also provides doctors, patients, and pharmacies the most efficient way to authorize, obtain, and fill prescriptions, all in a fully automated and reliable online network.

### SUMMARY OF THE INVENTION

The present invention provides a fresh method and system for truly automated online prescription purchases while providing improved security for issuing prescription simultaneously. Within the said online pharmacy network, both patients and doctors (M.D.s) are registered users. Doctors (M.D.s) will be verified by one of the authorities, such as the American Board of Medical Specialties (ABMS), an organization that maintains a database of physician board certification information on more than 700,000 specialty physicians representing 36 medical specialties and 88 subspecialties. ABMS contains information primary sourced from 24 recognized medical specialty boards, not self reported by physicians, and is fully recognized by key healthcare accreditation organizations for credentialing purposes. Once a doctor's status is verified, he/she has the authority to issue online prescriptions or approve patients' online prescription requests within the said online pharmacy network. Patients who wish to purchase prescription online will register with the said online pharmacy network and provide personal information such as credit card numbers, email addresses, and etc. Once a patient is registered with the said online pharmacy network, he/she has the ability to request an online prescription that will be used towards prescription purchases within the said online pharmacy network. A patient can choose from diverse payment methods and delivery preferences without obtaining or dropping off a conventional paper prescription at a pharmacy that has a physical location for pick-up. The present invention will reduce the likelihood of prescription abuse by avoiding counterfeit paper prescriptions, improve the efficiency of the prescription purchase by automating the processing of prescription writing and dropping off, permit patients who wishes to order discounted prescription drug online without adopting the existing prescription fax-in or mail-in methods.

### BRIEF DESCRIPTION OF THE DRAWINGS

FIG. 1 is an overall diagram of the system.

FIG. 2 is a high level flow diagram of the various operations that take place on patients' side.

FIG. 3 is a high level flow diagram of the various operations that take place on registered doctors' side.

### DESCRIPTION OF PREFERRED EMBODIMENTS

As used herein the following terms have the meaning given below:

1. “Doctors”—physicians, medical doctors, ones who are authorized to practice in the field of medical science and capable of issuing prescription pharmaceuticals.

2. “Prescription Prompt”—electronic prescription request made by the patients.

3. “System Controller”—means a computer system or web server that performs various calculations and operations hereinafter described.

4. “System Operator”—means an individual, company, party, entrepreneur or other entity that operates or is responsible for the computer system or web server that performs various calculations and operations hereinafter described. This entity will act as the liaison between doctors (M.D.s) and patients for obtaining electronic prescriptions.

### DETAILED DESCRIPTION OF THE INVENTION

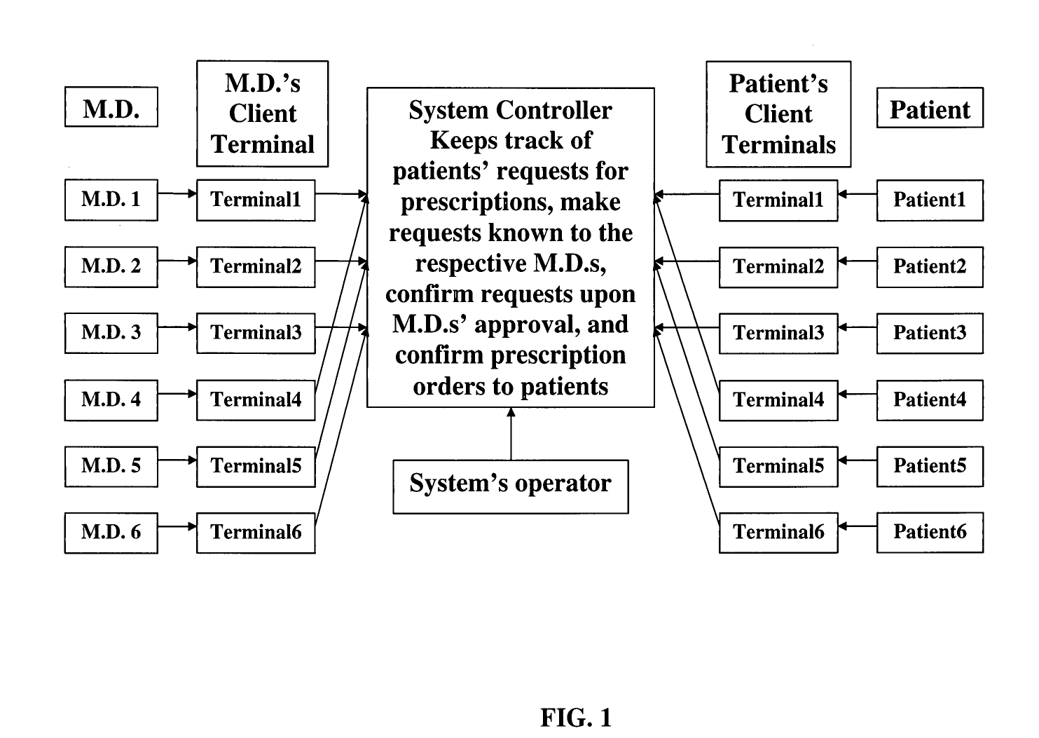

An overall diagram of the invention is shown in FIG. 1. The system connects the plurality of patients with plurality of doctors by means of a system controller. The actual numbers of doctors and patients can be greater or less than six. The system controller can handle almost unrestricted amount of prescription requests, prescription authorizations, and prescription redemption; at any time, there are plurality of patients, plurality of doctors, and plurality of prescription requests, authorizations, redemption, and purchasing within the system network.

The doctors communicate with the controller via the doctors' client terminals 1 to 6, and the patients communicate with the controller via patients' client terminals 1 to 6. Communication is via the Internet. Both the patients' and the doctors' terminals are connected to an ISP (Internet Service Provider) which provides access to the Internet. Correspondingly, controller is also connected to the Internet via an ISP. The drawing lines in FIG. 1 thus represent logical information flow and not physical connections. The doctors 1 to 6 and the patients 1 to 6 are described as being online.

The doctors' client terminals can be a variety of forms of terminals that are obtainable such as computers, laptops, WebTVs, PDAs, information appliances, or any other devices that can be used by the doctors to access the system controller over a network, so the doctors can issue prescriptions, authorize approval or decline any prescription requests from respective patients, and communicate with respective patients.

The system controller is one or more network servers running software to keep track the doctors' issued electronic prescription, patients' prescription prompts, doctors' authorizations or declines on respective prescription prompts; and correctly track or process prescription redemptions and purchases made by patients.

The system operator utilizes a client terminal to access and configure the system's controller as is conventional with computer systems and network servers.

The patients' client terminals are any of the various forms of terminals that are employed to access web sites such as computers, laptops, thin-client, WebTVs, two-way TV, PDAs, information appliances, or any other devices that patients may utilize to search for doctors within the network, request prescription approval, receive and view confirmation of approved prescription, redeem prescription within the said network, communicate with doctors, and be able to make purchases and payments using patients' client terminals.

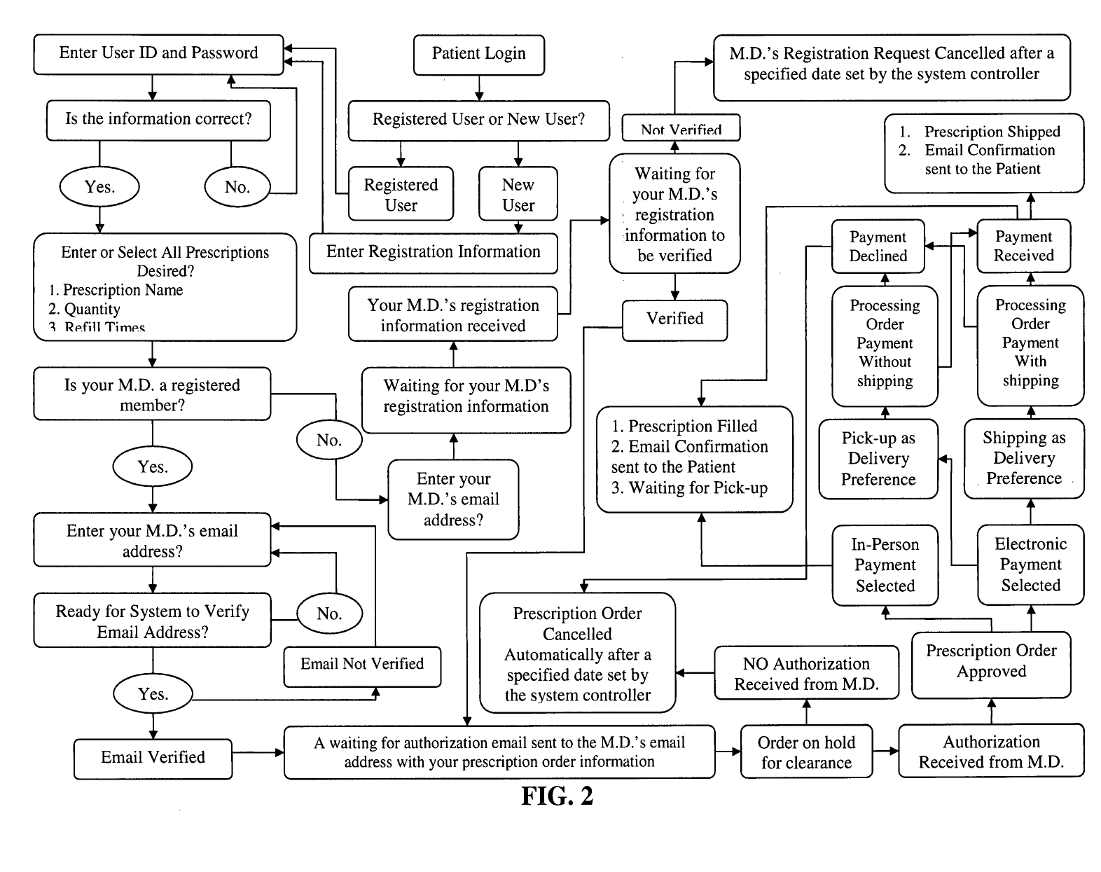

FIG. 2 is a high level flow diagram of the various operations taking place on the patients' end. Patient can begin the process of online prescription ordering by login to the online system network. If the patient is not yet a registered patient member of the said network, he/she will be required to do so. If the patient has already registered with the network, he/she will satisfy the login requirement and proceed to select the type of prescription needed. Once the prescription order is entered, the patient will be required to supply a doctor's email address, and the said doctor must be a registered member of the said prescription network. If the doctor of said patient's choice is not a registered physician member of the said prescription network, an email request to setup the membership is sent by the system controller to the doctor's email address. Upon recipient of the email request, the doctor will provide the system controller with registration information related to his/her field of specialty and ability to practice medical science, such as the doctor's certification information. This information will then be verified by one of the authorities, such as the American Board of Medical Specialties (ABMS), an organization that maintains a database of physician board certification information on more than 700,000 specialty physicians representing 36 medical specialties and 88 subspecialties. Upon confirmation from said medical authorities, the doctor's status within the pharmacy network will be confirmed as registered doctor. When a doctor is a registered member of the pharmacy network, he/she can issue prescriptions for his/her patients who are also registered patient member of the said pharmacy network. An email request for the above prescription will be sent to the respective doctor for approval, and the prescription order is put on hold. If such prescription order is not approved by the doctor within a set time period, the prescription order will be cancelled by the system controller. When a confirmation of approval is granted by the doctor, the patient will be notified by the system controller, and he/she can choose to either to redeem the prescription through an online payment or an in-person payment option. If electronic payment method is selected, the patient will select either pick-up or shipping as delivery preference. If the patient selects shipping as delivery preference, the patient's financial account will be charged of the amount of the prescription plus the applicable shipping costs, and a confirmation for prescription filled will be sent to the patient. If pick-up is selected, the patient's financial account will be charged of the amount of prescription, and the patient will be notified when the prescription is ready for pink-up. If in-person payment is selected, the patient will receive a notification upon prescription fill for pick-up.

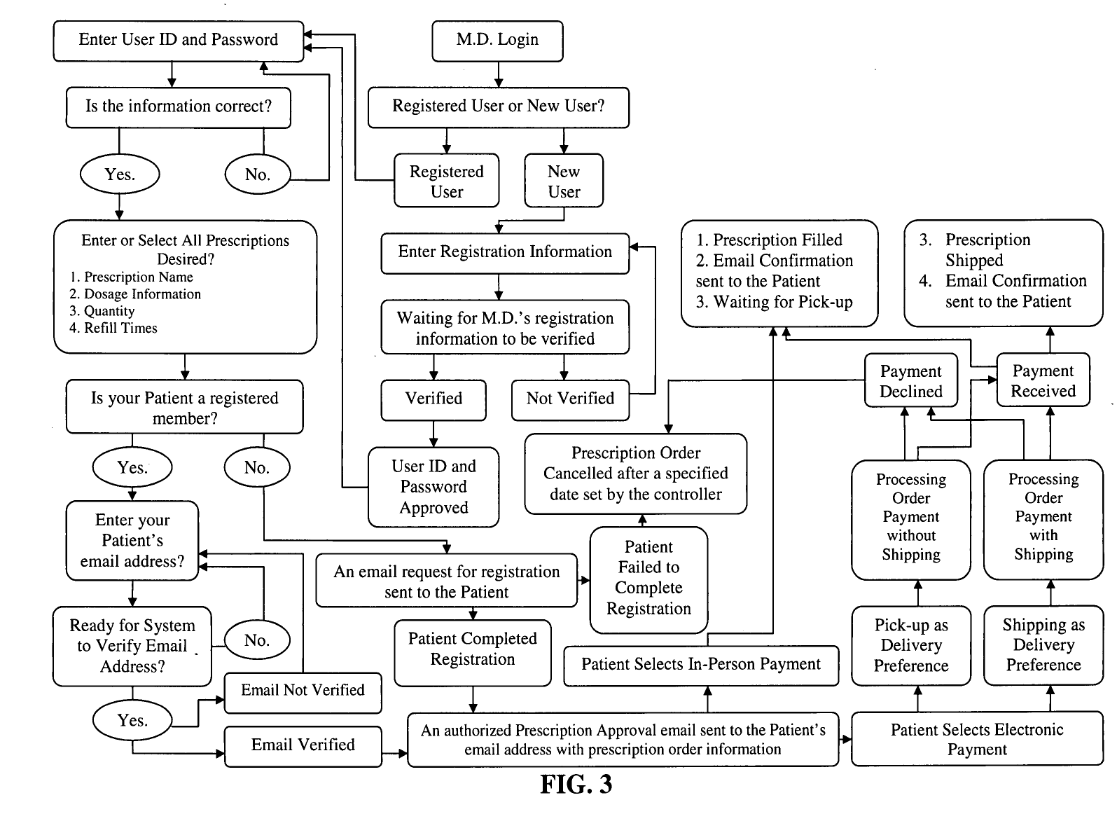

FIG. 3 is a high level flow diagram of the various operations taking place on the doctor's end. Patients are not the only ones who can prompt a prescription order, registered doctors can do the same on the said pharmacy network. After seeing a patient, a doctor can issue an authorized electronic prescription over the said pharmacy network for the patient's convenience. The doctor must also first register with the said pharmacy network and be confirmed by the medical authorities. The doctor can login to the pharmacy network and enter all information related to the patient's prescription. If the patient is not a registered patient member of the said pharmacy network, an email request for registration will be sent to the patient by the system controller upon a doctor's prescription prompt. If the patient chooses not to register, the electronic prescription will be invalidated by the system controller after a set time period. If the patient submitted his/her registration information to the pharmacy network, he/she will then be able to redeem the above mentioned prescription over the said pharmacy network. If the patient is already a registered patient member of the said pharmacy network, the doctor can simply enter the patient's email address, and a redemption notification of electronic prescription will be forwarded to the patient. If electronic payment method is selected, the patient will select either pick-up or shipping as delivery preference. If the patient selects shipping as delivery preference, the patient's financial account will be charged of the amount of the prescription plus the applicable shipping costs, and a confirmation for prescription filled will be sent to the patient. If pick-up is selected, the patient's financial account will be charged of the amount of prescription, and the patient will be notified when the prescription is ready for pink-up. If in-person payment is selected, the patient will receive a notification upon prescription fill for pick-up.

For instance, patient A has visited his physician and desired to purchase his medication online from a discounted pharmacy called XX. Given the available prescription processing method currently in the market, patient A will have to sent in his paper prescription, or the online pharmacy will call his doctor for verification, which leaves plenty room for prescription abuse since the pharmacy cannot accurately verify the person they contact is actually the physician. In either case, the operations taking place would only be categorized as inefficient and inconvenient. With the current invention, patient A, after visiting his physician, will instead request an electronic prescription to be issued at online pharmacy XX. To do so, his physician will join the pharmacy XX online network by providing information regarding his field of specialty and physician certificate. Pharmacy XX will then verify the physician's information with a medical authority, say American Board of Medical Specialties (ABMS). If the information supplied by the physician has been independently screened and confirmed by the ABMS, pharmacy XX will approve the physician for issuing electronic prescription that will be redeemed by patients who wish to use the service. The physician, after entering the pharmacy XX network, will enter medication needed by patient A, thus an electronic prescription is created for patient A for redemption. Patient A will enter the pharmacy XX network as a patient member, and make a purchase towards the electronic prescription. After electronic payment is received by pharmacy XX, a confirmation of shipment of the medication will be sent to patient A. Hence, the entire process from prescription issuing, redemption, purchasing, processing, and shipping will be streamlined to accomplish a truly automated online prescription purchase system that we have never seen in the past or at the present time.

Taking the above example, but assuming patient A needs the prescription quickly and wants to purchase prescription at his local pharmacy. Currently, many local pharmacies offer prescription drop off, but the patient still have to wait for the prescription to be filled. Even these pharmacies offer drive through, the patients will still have to spend idle time. No matter how much local pharmacies try to improve their operations, there is one thing they cannot alter—the need for prescription drop-off by the patient. The best approach so far adopted by the local pharmacies is calling of doctors (note that physicians never call pharmacies to accommodate their patients), which not only consumes additional human resource, but also fails to provide security measure to prevent possible prescription abuses. Though many local pharmacies have their online network, their prescription services are only limited to refills from past prescriptions issued by physicians. Following the above example, after visiting his physician, patient A subsequently requested an electronic prescription to be issued at his local pharmacy YY's online network. As usual, the physician provides information needed for verification by the medical authority such as ABMS, and he will receive a confirmation upon verification completion. The physician will issue an electronic prescription at the pharmacy YY's network, and patient A, will use his hand-held internet devices to make the redemption by choosing an electronic payment or preparing to make an in-person payment. When the prescription is filled, patient A will receive a confirmation for pick up, which could happen as the patient driving to his local pharmacy from his physician's clinic. Using the present invention, the step for prescription drop-off is eliminated, which provides efficiency and convenience to both local pharmacies and patients alike.

The current invention not only provides physician the fresh opportunity to issue electronic prescriptions, but also allows patients to make prescription prompt for approval by their physicians. Continue with the above example, assuming both patient A and his physician are registered members of pharmacy ZZ. Patient A enters the online pharmacy network ZZ and requests a prescription purchase for medication [12345], patient will supply with pharmacy ZZ with his physician's email address so that patient A's request for approval will be sent to his physician. His physician then schedules an appointment for patient A if he sees necessary, or the physician can simply respond to the request based on his knowledge about patient A's medical history if medication [12345] is a regular medication for patient A. After seeing patient A or coming to determination that such medication is needed even without an appointment, the physician will enter the pharmacy ZZ's network to give an electronic approval for the prescription. Pharmacy ZZ, after receiving confirmation from the physician, will notify patient A that his request is approved and is ready for redemption. Patient A will use this approved electronic prescription to purchase medication [12345] over pharmacy ZZ. Using the current invention, physicians, in their discretion, can issue patients (on regular medications) electronic prescriptions even without in-person visits.

The beauty of the current invention can be further demonstrated during time when physicians are not on duty. For example, patient A regularly uses medication [67890] to ease his slight asthmatic condition during fluctuating whether temperatures. During the evening hours when his physician is not present, the only way for patient A to obtain medication [67890] is through a visit to the emergency room, which is totally unnecessary and time consuming. With the present invention, patient A can communicate with his physician even after working hours though emails. At his discretion, physician can issue an electronic prescription at a 24-hour local pharmacy OO's online network, and patient A can redeem the prescription for immediate purchase of medication [67890] to ease his symptoms, a process which could not possibly be accomplished without the current invention.

Besides the advantages described above, the present invention shines in its ability to reduce potential prescription abuses. Currently, prescription pads can be purchased over the Internet with no verification procedures in place. Pharmacies, at the time accepting the paper prescriptions, do not check the validity of the prescriptions either due to the impracticality and unfeasibility of pharmacist-to-physician verification for each and every prescription order. In essence, anyone could possibly order prescription pads online with an actual doctor's identity. While many methods are being introduced for fancier and more secure prescription pads, the reality is that most physicians remain loyal to their old prescription pads in order to maintain low cost operations. The current invention provides a realistic means to achieve the objective of reducing prescription abuse by eliminating the need for physical prescriptions and bridging communications between physicians and pharmacists by utilizing a direct network. Using the current invention, prescription abuse can be effectively suppressed since the only way for anyone to obtain prescription is to have a physician issuing an electronic prescription that will be delivered directly and untainted by the system controller to the pharmacy, and the only one who can redeem the prescription at the pharmacy is the actual patient since the system controller keeps track of every prescription issued and the patient it was issued over its network.

With the rising prescription costs, many people are looking for pharmacies with the most competitive pricing. However, given the time and the complexity one must devote to redeem prescriptions online today, competitive pricing offered by many online pharmacies are overpowered by the need for convenience and simplicity that are the traits of local pharmacies. Hence, the tradeoff between costs and convenience are vividly evident in the pharmacy industry. With the current invention, patients will surpass the usual substitution between costs and convenience. Because the current invention is designed to modernize prescription issuing and redemption process, patients benefiting from this invention could enjoy both low cost medications online and have their online medication processed most efficiently ever. The current invention will produce and accelerate competitiveness in the pharmacy industry. When online pharmacies benefit from the current invention, local pharmacies will be compelled to implement changes such as lower prices on prescription pharmaceuticals in order to stay competitive in a fast paced environment.

Everyday, thousands of Americans crosses borders into Mexico and Canada for cheap medications, but many border pharmacies lack the reputation and safety measures for their in stock medications. The challenge is how to enhance safety measures for patients crossing borders so that their lives are well protected. So far, there is no regulation in place or agreements between U.S. and foreign governments to properly regulate the pharmacies along the U.S. borders, nor is there any control over the quality or safety of the pharmaceuticals produced and sold along the borders. The essential problem is that the American government has literally no control over foreign pharmaceutical sellers. However, the government could pass effective legislations to ensure patient's safety over U.S. based pharmaceutical distributors. Furthermore, direct government intervention was mostly costly and unproductive. With the current invention, electronic prescriptions are issued by verified physicians and processed at an unprecedented speed. Hence, instead of crossing borders, patients can order discounted medications with U.S. based pharmaceutical importing companies without delay. If the legislative body can precisely patrol and deploy penalties for importing companies that failed in the aspect of ensuring patient safety, these pharmaceutical importing companies themselves will exercise caution and diligence over the medications sold over its network in order to avoid penalties. Hence, another key application of the current invention is to promote purchasing of pharmaceuticals in domestic land to ensure an adequate level of safety precautions.

In summary, the present invention reduces the likelihood of prescription abuse, improves operational efficiency for pharmacies, elevates convenience level while reducing costs for patients, accelerates competitiveness in the pharmacy industry, enhances safety measures for patients, and simultaneously provides a viable approach for regulating pharmaceutical importing distributors instead of foreign distributors.

It should be understood that while various embodiments of the invention have been described, those skilled in art could make various changes in form, detail, and design without departing from the principle, spirit, and scope of the invention described herein. Applicant's invention is limited only by the scope of the appended claims.

We claim:

1. A method of conducting online transaction related to prescription authorization and redemption in which: a) a pharmacy network (retail based, online based, or combination of online and retail based) that has access to the web and to some degree utilize the Internet to operate its pharmacy business, where doctors, patients, pharmacies, and other classes of potential users are participants of the said network; b) doctors (M.D.s) who issue prescriptions will be verified by one of the authorities that can accurately confirm doctor's identity, field of specialties, license to practice in the field of medical science, and any other information that will substantiate a doctor's ability to practice and authorize prescriptions; c) when a doctor's status is confirmed by the said authorities, he/she will receive a confirmed status as a registered doctor within the said pharmacy network; d) a registered doctor's information such as name, user ID, password, license number, field of specialty, and any other information that will help identify and assure the doctor's status and ability to practice and authorize prescription will be stored by the pharmacy network; e) a registered doctor on the said network can authorize electronic prescription online, or to confirm or decline any electronic prescription requests from his/her patients who are registered patient members of the said pharmacy network; f) a patient's information such as name, age, gender, user ID, password, email address, and etc. will be stored by the said pharmacy network; g) a patient can enter a prescription request and have the system controller to send out the request to the patient's specified doctor who is a registered doctor member of the said pharmacy network for approval; h) If the prescription request is declined by the patient's doctor, then the pending prescription order will be cancelled by the system controller of the said pharmacy network; i) If the prescription request is approved by the doctor, a confirmation will be sent to the patient for prescription redemption over the said pharmacy network.

2. A method of conducting online transaction related to prescription purchasing and delivery in connection with said method as in claim 1 in which: a) the patient will have the option to choose among diverse prescription medicine delivery methods; b) if the patient chooses to pick up the prescription medicine, he/she will be notified by the said pharmacy network when the prescription is ready for pick up; c) if the patient chooses to have the prescription medicine shipped, he/she will choose from different shipping methods and will have to make an electronic payment in advance for the purchase and shipment of the prescription medicine; d) if the patient's payment for the prescription medicine is cleared, then the patient will be notified by the system controller of the status of the shipment; e) if the patient's payment is declined, then a new request for payment will be sent to the patient by the system controller; f) if the payment is not cleared after a specified time set by the controller, the prescription order will be cancelled automatically.

3. A method as in claim 1 wherein said transaction is an online transaction.

4. A method as in claim 1 wherein said transaction is for goods and services, more specifically electronic version of prescriptions and electronic prescription issuing and authorizing services.

5. A method as in claim 1 wherein said transaction is for goods, more specifically pharmaceutical products that require doctor's prescription prior to purchase.

6. A method as in claim 2 wherein said transaction is an online or offline transaction.

7. A method as in claim 2 wherein said transaction is for goods and services, more specifically electronic version of prescriptions' redemption, purchasing and delivery services.

8. A method as in claim 2 wherein said transaction is for goods, more specifically pharmaceutical products that require doctor's prescription prior to purchase.

9. An online system for conducting online prescription issuing, redemption, purchasing, delivery, the system comprising:
a) A controller;
b) Doctors' terminals which communicate with said controller whereby doctors can enter and transmit, a prescription or prescription approval for said at least one prescription pharmaceutical goods, a prescription comprising the drug name, quantity, refill times, dosage, and any other prescription information associated with the said prescription pharmaceutical goods;
c) Web pages which display said electronic prescriptions and prescription approval requests;
d) Patients' terminals which communicate with said controller whereby said patients can select specific doctors for authorizations of electronic prescriptions, enter and transmit requests for electronic prescriptions pertaining to at least one prescription pharmaceutical goods, a request comprising the doctors' contact information such as email addresses and user IDs, drug name, quantity, refill times, dosage information, and any other prescription information associated with the prescription pharmaceutical goods;
e) Said controller relates patients' requests for electronic prescriptions to their respective doctors displaying information identify respective patients and respective prescription requests to their respective doctors;
h) An email system which operates on said computer server to notify said doctors and said patients any prescription issuing, prescription approval or prescription request, and confirmations related to said prescription pharmaceutical goods; and the said email system allows communications between said doctors and said patients regarding said prescription pharmaceutical goods;
i) A database system which operates on said computer server to store information or data such as user IDs, passwords, email addresses, contact information, doctor license numbers, filed of specialties, prescription order history, credit card numbers, debit card numbers, banking account numbers, and all information related to prescription requesting, prescription issuing, prescription redemption, prescription purchasing, prescription order processing, prescription shipping, and payment processing;

10. The system recited in claim 9 operating in accordance with said specified and limited time period whereby doctors' prescription approval and patients' prescription request for prescription pharmaceutical goods are conducted during said specified and limited time period.

11. The system recited in claim 9 wherein said doctors' terminals are terminals connected to the Internet.

12. The system recited in claim 9 wherein said patients' terminals are terminals connected to the Internet.

13. The system recited in claim 9 wherein said controller comprises a server.

14. The system recited in claim 9 wherein said doctors' terminals and said patients' terminals communicate with said controller via the Internet.
